SEQUENCE LISTING (2 sequences)

SEQ ID NO: 1 - protein, 312 aa, Streptococcus pyogenes
DSFSANQEIRYSEVTPYHVTSVWTKGVTPPAKFTQGEDVFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 2 - protein, 341 aa, Streptococcus pyogenes
MRKRCYSTSAVVLAAVTLFALSVDRGVIADSFSANQEIRYSEVTPYHVTSVWTKGVTPPAKFTQGEDVFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

(19) United States
(12) Patent Application Publication
(10) Pub. No.: US 2024/0425838 A1
(43) Pub. Date: Dec. 26, 2024

(54) TREATMENT OF ADVANCED IgA NEPHROPATHY

(71) Applicant: Bengt FELLSTRÖM, Knivsta (SE)

(72) Inventor: Bengt FELLSTRÖM, Knivsta (SE)

(21) Appl. No.: 18/706,132

(22) PCT Filed: Nov. 10, 2022
(86) PCT No.: PCT/SE2022/051047

§ 371 Date: Apr. 30, 2024

(30) Foreign Application Priority Data
Nov. 10, 2021 (EP) 21207349.8

(51) Int. Cl. C12N 9/64; A61K 38/00

(52) U.S. Cl. CPC C12N 9/6472; A61K 38/00

(57) ABSTRACT

The present invention provides IdeS polypeptide for use in a method of treating or pre-venting a disease mediated by pathogenic polymeric IgA complexes. wherein said dis-ease or disorder is progressive IgA nephropathy (IgAN) or recurrent IgAN following re-nal transplantation. The IdeS polypeptides finds particular use in induction therapy of progressive or recurrent IgAN disease or disorder.

### FIELD OF THE INVENTION

The present invention relates to the fields of medical science, immunology and renal diseases. The present invention provides compounds for use in the treatment of progressive IgA nephropathy or recurrent IgA nephropathy following renal transplantation and uses of such compounds to disintegrate pathogenic immune complexes comprising IgA molecules.

### BACKGROUND OF THE INVENTION

Immunoglobulin A (IgA), one of the five primary immunoglobulins, plays a pivotal role in mucosal homeostasis in the gastrointestinal, respiratory, and genitourinary tracts, functioning as the dominant antibody of immunity in this role. It is the second most abundant immunoglobulin type found in the body and, consequently, has a crucial role in protection against antigens. IgA nephropathy (IgAN), previously known as Berger's disease, is the most common global primary glomerular disease that can progress to renal failure. It is a disease of young adults, with a peak incidence in patients in their 20s and 30s. (Berthoux F C, et al. Natural history of primary IgA nephropathy. Semin Nephrol. 2008;28(1):4-9; Wyatt R J, et al. IgA nephropathy. N Engl J Med. 2013;368:2402-2414; Zhang et al. Immunoglobulin A nephropathy: current progress and future directions. Transl Res. 2015;166(2):134-44.). The incidence of IgAN is greatest in East and SouthEast Asian populations. Of all patients on dialysis treatment roughly 12-15% have developed end stage renal failure (ESRD) because of IgAN in the western world, and corresponding numbers are much higher, 30-40%, in Asia. The prevalence of IgAN is also much higher in Asia than in Europe or USA. IgA nephropathy is a lifelong disease leading to CKD and progresses to ESRD in 30%-40% of patients over the course of 15-20 years. Following renal transplantation (RTx) roughly 50% will show IgA deposition histologically and eventually 15% will lose their graft because of recurrence of IgAN in the transplant. (Berthelot, et al. Recurrent IgA nephropathy is predicted by altered glycosylated IgA, autoantibodies and soluble CD89 complexes. Kidney International (2015) 88, 815-822). Patients initially present with hematuria and hypertension and proteinuria develops as the disease progresses. Diagnosis of IgAN is made by renal biopsy demonstrating IgA containing deposits in the glomeruli, frequently associated with complement 3 (C3).

The pathophysiology of IgAN is related to the overproduction of poorly-galactosylated immunoglobulin A1 (IgA1), which accumulates in the kidney glomeruli. However, changes in galactosylation alone is insufficient to induce renal injury; the participation of glycan-specific IgG autoantibodies that recognize the poorly-galactosylated IgA1 molecule is required. This process leads to local inflammation and complement activation in the kidney. Both the alternate and lectin complement pathways may be activated, leading to generation of anaphylatoxins, membrane attack complex (MAC) and terminal complement (C5b-9), with subsequent promotion of inflammatory mediators in the glomeruli. The inflammatory reaction leads eventually to remodeling and destructive processes involving MC proliferation and activation, with consequential scarring and non-functional nephrons (FIGS. 1-2).

Present treatment options for IgAN includes renin-angiotensin system (RAS) blocking agents, such as angiotensin-converting enzyme (ACE) inhibitors or angiotensin receptor blockers (ARB). These therapies are aimed at controlling blood pressure, preserving kidney function through decreasing intraglomerular pressure, which in turn reduces proteinuria. Patients with baseline hypertension and proteinuria>1 g/day are at increased risk for progression. These treatments are insufficient in preserving renal function as the proportion of patients who progress to CKD and ESRD are high. In those patients who remain at high risk of progression despite optimal treatment with ACE or ARB our guidelines (KDIGO) stipulate that a course of corticosteroids may be considered or the patient should be offered enrollment into a clinical trial. In rapid progressors heavy immuno-suppression may be offered, at the expense of serious side-effects.

In recurrence of IgAN in RTx, there is no treatment available to stop or attenuate the gradual damage occurring due to the recurrent disease. At present there are a number of ongoing clinical trials, targeting proteinuria primarily, but with the μltimate goal to stabilize renal function and prevent development of ESRD. Examples are a handful of complement inhibitors, NEFECON, sparsentan etc, all on top of optimized RAS-blockade. Rituximab was tried but did not quite make it.

In most patients the progression to renal failure is a rather slow process, but there are also IgAN cases progressing much more rapidly, characterized by significant proteinuria and loss of GFR>5 ml/min/year. These patients often present with elevated concentrations of anti-glycan (IgA1) IgG, IgG-GdIgA1 immune complexes (IC) and complement activation. They are difficult to treat and seldom respond to traditional therapies including cyclophosphamide, high doses of corticosteroids and plasmapheresis. It has also been shown that patients with recurrence of IgAN have elevated concentrations of IgG-GdIgA1 ICs.

WO2021160805 relates to methods for detecting IgM, IgA and IgE antibodies and new therapies for diseases and conditions mediated by pathogenic antibodies and antibody complexes involving the use of an IgG cysteine protease. WO2021160805 mentions IgAN in general as a condition involving pathogenic antibodies but does not make any distinction between different stages of IgAN, or other conditions involving the formation of immune complexes comprising complexes of IgA, that results in the formation of pathogenic immune complexes as compared to non-pathogenic immune complexes. The application has a particular focus on IgG and IgM and merely confirms that an IgG cysteine protease is capable of cleaving IgG molecules when present in a complex with IgM.

Hence, there is a need for an effective and safe new treatment option for stages of IgAN associated with pathogenic and reactive immune complexes comprising IgA, particularly for progressive IgAN and recurrent IgAN following renal transplantation.

### SUMMARY OF THE INVENTION

The above stated problem has now been solved or at least mitigated by the provision herein of a new medical use of an IdeS polypeptide.

In this regard, it has surprisingly been found that IdeS is capable of effectively disintegrating polymeric IgA complexes which are pathogenic into non-pathogenic parts.

It has further been found that IdeS reduces the ability of IgA immune complexes to activate human mesangial cells, demonstrating a clear potential for the use of IdeS in the treatment of IgA nephropathy (IgAN), more specifically in more advanced stages of IgAN involving large immune complexes comprising a large amount of IgA1 molecules.

Specifically, it has for the first time been shown herein that when such polymeric IgA complexes are disintegrated by the use of an IdeS polypeptide they will turn into monomeric IgA1 molecules, which are not reactive to mesangial cells in vitro any more. This adds a further unexpected dimension to the IgG cleaving capability of IdeS already known in the art.

The proposed mechanisms for IgA nephropathy and a schematic illustration of the creation of poorly galactosylated polymeric IgA1 resulting in immune complex formation and mesangial deposition in the kidney are shown in FIGS. 1 and 2 respectively.

More importantly, IdeS was shown herein to be capable of neutralizing the negative effects of the high-density IgA immune complexes comprising a large amount of IgA molecules, thereby paving the way for IdeS for use in the treatment or prevention of progressive or recurrent IgAN following renal transplantation, where the presence of such complexes are of high relevance for the progress and severity of the disease. Such a use of an IdeS polypeptide has not previously been disclosed in the art.

It was surprisingly shown herein that IdeS is capable of neutralizing large amounts of complexes by one induction meaning that it will be useful as an induction therapy in acute stages of IgAN.

Accordingly, in a first aspect the present invention provides an IdeS polypeptide for use in a method of treating or preventing a disease or disorder mediated by polymeric IgA complexes in a subject, wherein said disease or disorder is progressive IgA nephropathy (IgAN) or recurrent IgAN following renal transplantation. The disease or disorder is mediated mainly by pathogenic polymeric IgA complexes as further explained herein. This may be referred to herein as a disease or disorder mediated by pathogenic polymeric IgA complexes, even if other parameters may also be involved.

In a second aspect the present invention provides a method for treating or preventing a disease or disorder mediated by polymeric IgA complexes, wherein said disease or disorder is progressive IgA nephropathy (IgAN) or recurrent IgAN following renal transplantation, which method comprises the administration of an effective amount of an IdeS polypeptide to a subject in need thereof.

### BRIEF DESCRIPTION OF THE DRAWINGS

FIG. 1. 4-hit pathogenesis model of IgA nephropathy. 1: Hit 1—Increased circulating galactose-deficient IgA1. 2: Hit 2—Production of unique anti-glycan antibodies. 3: Hit 3—Formation of pathogenic IgA1-containing circulating immune complexes. 4: Hit 4—Mesangial deposition and activation of mesangial cells resulting in glomerular injury. 5: Proliferation, ECM production, Cytokines, Growth factors. 6: Mesangial cell. 7: IgA1complexes. C: Cytokines. P: Podocyte.

FIG. 2. Schematic illustration of the creation of poorly galactosylated polymeric IgA1 resulting in immune complex formation and mesangial deposition in the kidney. L: Lumen. MI: Mucosal infection. M: Mucosa. C: Cytokines. CS: Systemic circulation. GB: Genetic background. IR: Immune response. 2: IgA⁺ ASC mistrafficking (MT) to systemic circulation. 3: Secretion of poorly galactosylated polymeric IgA1. 5a: IgG+ IgA1 autoantibodies to IgA1 hinge region. 5b: Cross-reactive antimicrobial antibodies. 5c: sCD89 shedding. 5: Immune complex formation. 6: Mesangial deposition. RI: Renal injury.

FIG. 3. IgG immunoblot of the non-reduced IgA IdeS digests. To the left of the molecular weight ladder: 1 ml of purified IgA (1 mg/ml) was incubated with 5 μg of IdeS for 5, 15, 30, 60 & 80 minutes. To the right of the molecular weight ladder: 25 μg, 55 μg and 60 μg of IdeS were added to 1 ml aliquots of purified IgA (1 mg/ml) for 80 minutes at room temperature. (2.5% BSA blocking solution, polyclonal rabbit anti human IgG HRP 1:2000 dilution). A: Polymeric IgG. B: F(ab′)2. C: Fragment of the gamma heavy chain.

FIG. 4. IgA immunoblot of the non-reduced IgA IdeS digests. To the left of the molecular weight ladder: 1 ml of purified IgA (1 mg/ml) was incubated with 5 μg of IdeS for 5, 15, 30, 60 & 80 minutes. To the right of the molecular weight ladder: 25 μg, 55 μg and 60 μg of IdeS were added to 1 ml aliquots of purified IgA (1 mg/ml) for 80 minutes at room temperature. (2.5% BSA blocking solution, anti human IgA HRP 1:1000 dilution).

FIG. 5. Western blot analysis of reduced IgA IdeS digests. To the left of the molecular weight ladder: 1 ml of purified IgA (1 mg/ml) was incubated with 5 μg of IdeS for 5, 15, 30, 60 & 80 minutes. To the right of the molecular weight ladder: 25 μg, 55 μg and 60 μg of IdeS were added to 1 ml aliquots of purified IgA (1 mg/ml) for 80 minutes at room temperature. (2.5% BSA blocking solution, polyclonal rabbit anti human IgG HRP 1:2000 dilution). Fu: Full gamma-heavy chain. Fr: Fragment of the gamma heavy chain.

FIG. 6. Densitometric analysis of the gamma heavy chain band present in IgA IdeS digests. To the left: 1 ml of purified IgA (1 mg/ml) was incubated with 5 μg of IdeS for 5, 15, 30, 60 & 80 minutes. To the right: 25 μg, 55 μg and 60 μg of IdeS were added to 1 ml aliquots of purified IgA (1 mg/ml) for 80 minutes at room temperature.

FIG. 7. Elution profile of intact IgA following gel filtration by FPLC using a HiLoad 16/600 Superdex 200 pg FPLC column.

FIG. 8. Elution profile of IdeS digested IgA following gel filtration by FPLC using a HiLoad 16/600 Superdex 200 pg FPLC column.

FIG. 9: Overlayed elution profiles of intact and IdeS digested IgA following gel filtration by FPLC using a HiLoad 16/600 Superdex 200 pg FPLC column.

FIGS. 10A and 10B. IL-6 generation by two different human mesangial cell lines following exposure to IgA samples (100 μg/ml) undigested and digested with 60 μg of IdeS.

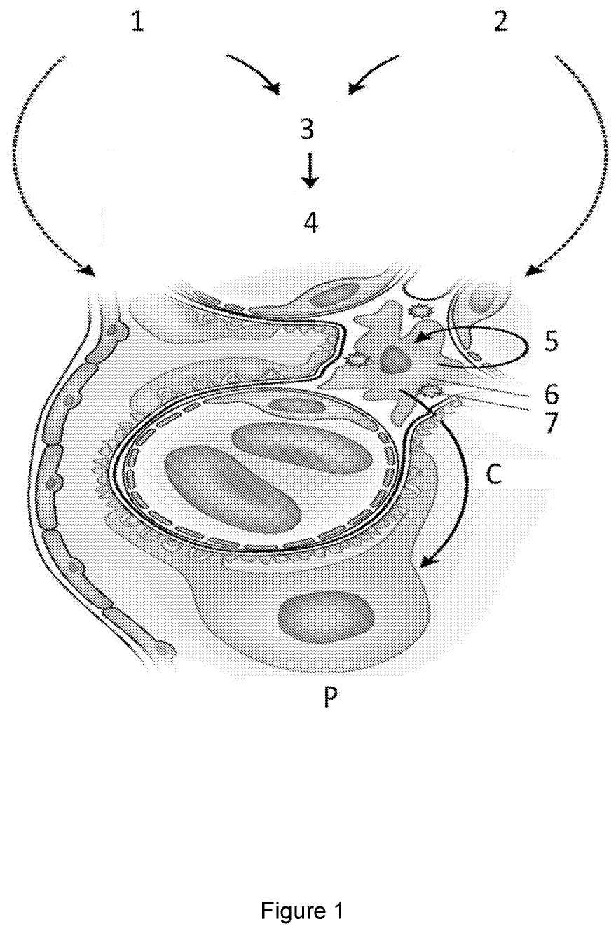

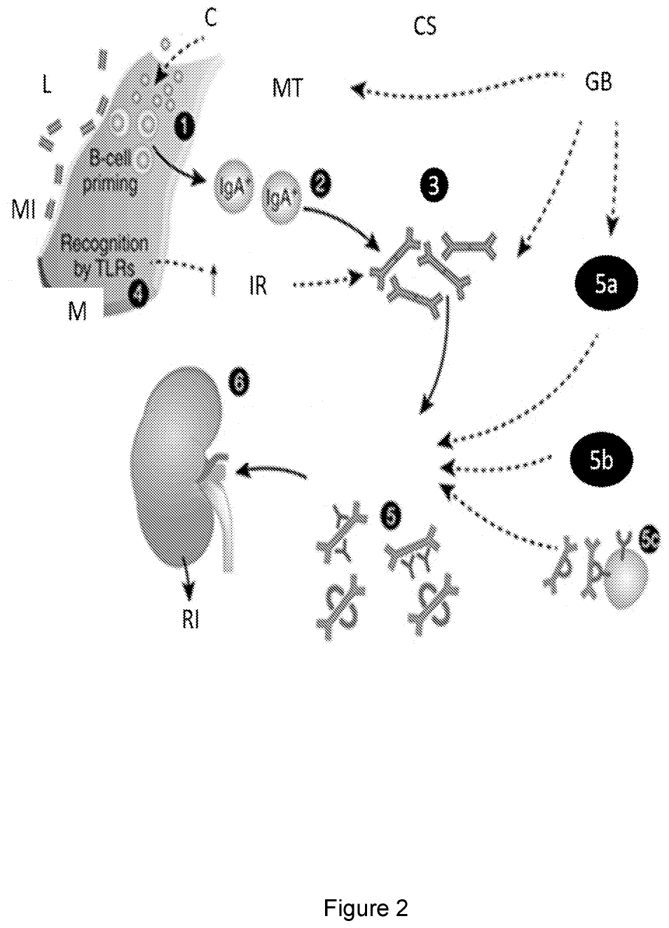

### DESCRIPTION OF THE INVENTION

### Definitions

Details of the present invention are set forth below. Although any materials and methods similar or equivalent to those described herein can be used in the practice or testing of the present invention, the preferred materials and methods are now described.

Other features, objects and advantages of the invention will be apparent from the description. In the description, the singular forms also include the plural unless the context clearly dictates otherwise.

Unless defined otherwise, all technical and scientific terms used herein have the same meaning as commonly understood by one of ordinary skill in the art to which this invention belongs.

The terms “protein” and “peptide” should be construed to have their usual meaning in the art.

The term “subject” may be used interchangeably herein with the terms “individual”, “patient” and the like.

The terms “treatment” or therapy”, or the like, may be used interchangeably herein.

The Immunoglobulin-degrading Enzyme from Streptococcus pyogenes (IdeS) is a highly specific protease that cleaves Immunoglobulin G (IgG) at a single site below the hinge region, yielding F(ab′)₂ and Fc fragments. The IdeS protease has been used i.a. to characterize antibodies using liquid chromatography-mass spectrometry (LC/MS). When combined with PNGase F, IdeS will fragment and deglycosylate antibodies in a single step. IdeS has also been used for therapeutic purposes, namely as imlifidase for the treatment of desensitization of highly sensitized adults needing kidney transplantation (EP1901773B1).

IdeS is commercially available from e.g. Promega and as the product Idefirix.

The sequence of IdeS from Streptococcus pyogenes
is SEQ ID No: 1:
DSFSANQEIRYSEVTPYHVTSVWTKGVTPPAKFTQGEDVFHAPYVANQGW
YDITKTFNGK
DDLLCGAATAGNMLHWWFDQNKEKIEAYLKKHPDKQKIMFGDQELLDVRK
VINTKGDQTN
SELFNYFRDKAFPGLSARRIGVMPDLVLDMFINGYYLNVYKTQTTDVNRT
YQEKDRRGGI
FDAVFTRGDQSKLLTSRHDFKEKNLKEISDLIKKELTEGKALGLSHTYAN
VRINHVINLW
GADFDSNGNLKAIYVTDSDSNASIGMKKYFVGVNSAGKVAISAKEIKEDN
IGAQVLGLFT
LSTGQDSWNQTN
The full sequence of IdeS including the signal
peptide from Streptococcus pyogenes is
SEQ ID No: 2:
MRKRCYSTSA VVLAAVTLFA LSVDRGVIAD SFSANQEIRY
SEVTPYHVTS VWTKGVTPPA KFTQGEDVFH APYVANQGWY
DITKTFNGKD DLLCGAATAG NMLHWWFDQN KEKIEAYLKK
HPDKQKIMFG DQELLDVRKV INTKGDQTNS ELFNYFRDKA
FPGLSARRIG VMPDLVLDMF INGYYLNVYK TQTTDVNRTY
QEKDRRGGIF DAVFTRGDQS KLLTSRHDFK EKNLKEISDL
IKKELTEGKA LGLSHTYANV RINHVINLWG ADFDSNGNLK
AIYVTDSDSN ASIGMKKYFV GVNSAGKVAI SAKEIKEDNI
GAQVLGLFTL STGQDSWNQT N

The term “IdeS polypeptide” as used herein is intended to mean a cysteine proteinase which cleaves IgG with substantially the same degree of specificity as the IdeS from Streptococcus pyogenes (SEQ ID NO:1), see e.g. Wenig et al, PNAS (2004):101:17371-17376. An IdeS polypeptide may therefore be SEQ ID NO:1 or a variant thereof, such as an analogue a fragment or an extended variant thereof. Such variant and analogues are known in the art and they are often used in order to provide polypeptides having one or more improved properties, such as activity, stability, solubility, immunogenicity and the like. Cysteine proteases that are functionally equivalent to IdeS are also envisaged within the context of the present disclosure.

Herein the term “IdeS polypeptide” may be used interchangeably with the term “IdeS”.

Herein any suitable source of an IdeS polypeptide, such as exemplified herein, may be used to provide an IdeS polypeptide to a subject in question in the context of the present invention. There are also envisaged herein functional fragments or variants of an IdeS polypeptide as described elsewhere herein.

The present disclosure has for the first time demonstrated the effect of an IdeS polypeptide on pathogenic immune complexes comprising IgA1 molecules, and that these complexes, once disintegrated, alter their properties into a non-pathogenic behavior. Disease conditions involving IgA molecules in immune complexes are of a very specific nature and this is the first time that a reducing effect on their pathogenicity has been proven.

Immune complexes comprising IgA1 and IgG molecules are occurring in various amounts both in healthy individuals and in individuals with IgA nephritis (IgAN). However, no one has previously been able to make the distinction and present a treatment or prevention of stages of IgAN where the complexes have become many, large and pathogenic, such as in progressive or recurrent IgAN disease.

IdeS, or IgG cysteine proteases, previously known to be able to cleave IgG molecules were not expected to have such a strong reducing effect on the pathogenicity of IgA molecules when present in large immune complexes. In such immune complexes the number of IgA molecules is high which in particular is the case in advanced stages of IgAN or recurrent IgAN disease following renal transplantation.

Accordingly, patients with progressive IgAN disease or recurrent IgAN, such as recurrent IgAN in renal transplant do have distinctly increased levels of immune complexes, which are often polymeric IgA. These are pathogenic and stimulate renal mesangial cells to become activated and proliferate with subsequent formation of inflammatory proteins, growth factors and complement activation.

Eventually this leads to remodeling of glomeruli followed by fibrosis formation and nephron deterioration, including that glomeruli will enter into necrosis and become nonfunctional. Herein, we have demonstrated that if these polymeric IgA complexes are disintegrated by the use of IdeS they will turn into monomeric IgA1 molecules, which are not reactive to mesangial cells in vitro any more.

This presents the surprising use of the IdeS polypeptide in late-stage and acute IgAN disease. In summary, it is only reactive polymeric IgA1 containing immune complexes that are harmful and pathogenic and cause diseases like progressive IgAN or recurrent disease following renal transplants, and not any other stage of IgAN not including such reactive complexes.

More specifically, it is herein presented for the first time IdeS for use in a method of treating or preventing a disease or disorder mediated by pathogenic polymeric IgA complexes in a subject, wherein said disease is progressive IgA nephropathy (IgAN) or recurrent IgAN following renal transplantation.

There is also provided IdeS for use in a method of treating or preventing a disease or disorder mediated by pathogenic polymeric IgA complexes in a subject, wherein said disease is progressive IgA nephropathy (IgAN).

There is also provided IdeS for use in a method of treating or preventing a disease or disorder mediated by pathogenic polymeric IgA complexes in a subject, wherein said disease is recurrent IgAN following renal transplantation.

The surprising finding of the effect of IdeS on rapidly progressing IgAN indicates a lower threshold for using IdeS in IgAN. In turn, this presents a new way of resetting immune complexes comprising IgA in the circulation and in the kidney thereby presenting the use of IdeS as an induction therapy in advanced, i.e. late-stage or recurrent stages of IgA, wherein said subject may be an acute incident subject.

Accordingly, in the first aspect the present invention provides an IdeS polypeptide for use in a method of treating or preventing a disease mediated by pathogenic polymeric IgA complexes, wherein said disease or disorder is progressive IgA nephropathy (IgAN) or recurrent IgAN following renal transplantation.

There is also provided the use of an IdeS polypeptide in the manufacture of a medicament for treating or preventing a disease mediated by pathogenic polymeric IgA complexes, wherein said disease or disorder is progressive IgA nephropathy (IgAN) or recurrent IgAN following renal transplant.

There is furthermore provided herein the IdeS polypeptide for use, wherein said method comprises using IdeS as an induction therapy of said IgAN disease or disorder, wherein said induction treatment is followed by a subsequent one or more additional treatment(s) comprising an IdeS polypeptide and/or other IgAN specific treatments.

An “induction treatment” or “induction therapy”, which terms may be used interchangeably herein, is the first in a series of therapeutic measures taken to treat a disease or disorder. This initial treatment or therapy is designed to bring about a remission of a condition. In the present context, this means that the first treatment with an IdeS polypeptide in essence is envisaged to clear pathogenic IgA containing complexes from the body of the subject and that the treatment following this induction therapy is intended to mainly maintain the clearance of the complexes in the body. Hence an induction therapy needs to be strong and efficient, and it needs to have the ability to set back the main drivers of the disease or disorder. Most likely an induction therapy is needed to be followed by maintenance treatment using traditional IgAN treatment or immunosuppression, or other treatments as mentioned elsewhere herein.

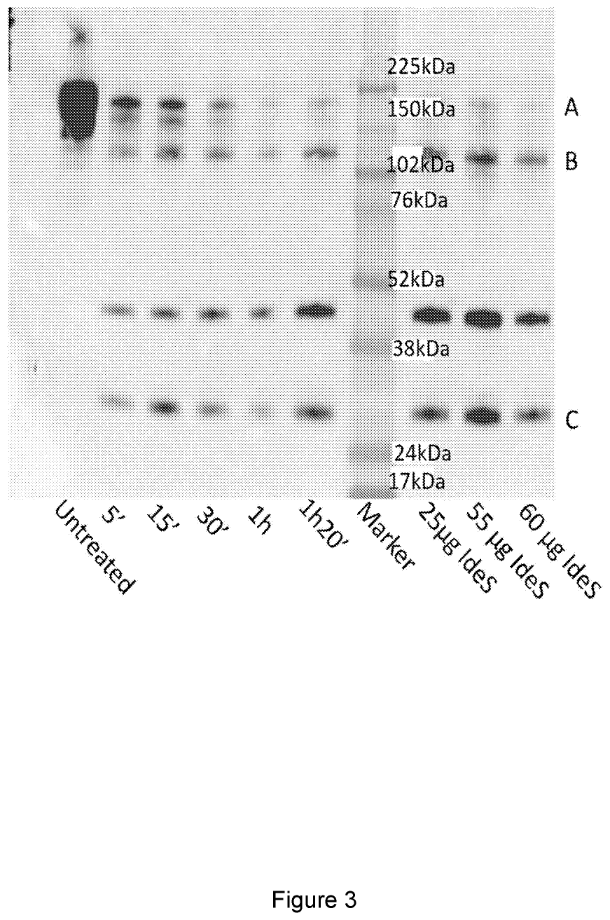

This surprising effect of an IdeS polypeptide is illustrated e.g. in example 1, FIG. 3, where digestion with IdeS resulted in a decrease in the very high molecular weight IgG complexes in patients with an acute incident IgAN both in a time and concentration-dependent manner. These acute incident patients possessed IgG mainly in the form of high and very high molecular weight complexes, presumably complexed with IgA, which is the general situation for patients in a condition of progressive or recurrent IgAN.

Preliminary additional data also shows that IdeS treatment of patients leads to a rapid and substantial (almost total), reduction of circulating IgA containing immune complexes (data not shown).

That the IdeS polypeptide is so effective in this patient group was very unexpected. These results mirror the possibility of clearing the circulation of pathogenic IgA complexes in said patients, thereby presenting a use in induction therapy. This is a significant improvement to the methods used in the art. As an example, many of the previous methods are insufficient in preserving renal function and the proportion of such patients progressing to CKD and ESRD is high. In addition, serious side effects are many and often occurring within existing treatments.

The time required for a treatment with an IdeS polypeptide may comprise days, months or years or rather for as long as the condition of the patient so requires. The dosage regimen will be dependent on each individual and stage of disease and will be determined by the skilled person.

Herein, said IgAN specific treatment may be selected from the group consisting of Renin-angiotensin system (RAS) blockage, corticosteroids such as budesonide, pulse therapy comprising cytostatic compounds, immunosuppressive treatments, such as immunosuppressive treatments comprising mycophenolate mofetil (MMF), calcineurine inhibitors, anti-CD20 antibodies and other treatments targeting IgG immune complex formation, such as treatments comprising FcRN interactions to prevent the formation of IgG immune complexes.

Said other IgAN specific treatment may also comprise using an IdeS polypeptide in the form of an mRNA-based therapeutic agent encoding said IdeS polypeptide.

Hence, such an mRNA based therapeutic is envisaged to be used in combination with an induction treatment with an IdeS polypeptide as described elsewhere herein, wherein said induction treatment comprises the administration to a subject in need thereof of an IdeS polypeptide per se subsequently followed by the administration of an IdeS polypeptide in the form of an mRNA-based therapeutic agent encoding said IdeS polypeptide. This is an alternative, or may be used in combination with, other IgAN specific treatments mentioned elsewhere herein.

There is also provided an Ides polypeptide for use as disclosed herein, wherein said subject is an acute incident subject. This means that said subject is at an acute stage of their condition therefore in need of an effective induction therapy for an initial clearance or remission of the condition, as discussed elsewhere herein.

Said subject as mentioned herein may also be at risk of developing Chronic Kidney Disease (CKD) or End Stage Renal Failure (ESRD).

There is also provided herein an IdeS polypeptide comprising an amino acid sequence according to SEQ ID NO:1, or a functional fragment or variant thereof. A functional fragment or variant thereof possesses the characteristics of IdeS as defined elsewhere herein but may contain minor modifications in the amino acid sequence to provide equivalent or improved functionality to the enzyme.

There is also provided herein an IdeS polypeptide for use, wherein said IdeS polypeptide is provided to said subject in the form of an mRNA-based therapeutic agent encoding said IdeS polypeptide.

An IdeS polypeptide may be administered as a protein or peptide therapeutic agent. It is also envisaged that an IdeS polypeptide as disclosed herein may be administered indirectly, such as by an expression vehicle encoding said enzyme where the enzyme is only expressed and thereby made functional once it has been administered to a subject and entered the body of the subject, where it subsequently may be expressed. An example of an expression vehicle is a DNA- or an RNA based vector, or variants thereof, encoding said IdeS polypeptide further comprising functional units for transcribing and/or expressing the polypeptide in said subject. These are technologies well-known in the art.

In this regard, it is envisaged that an IdeS polypeptide may be administered as an mRNA-based therapeutic agent encoding said IdeS polypeptide. Accordingly, there is provided herein a composition comprising an mRNA encoding an IdeS polypeptide optionally in combination with a pharmaceutically acceptable carrier. When the mRNA has been administered to a subject in need thereof, it is translated to produce the polypeptide of interest, i.e. an IdeS polypeptide in the body of said subject.

Methods for producing mRNA agents are known in the art and are described e.g. in. Hassett K J, et al., Impact of lipid nanoparticle size on mRNA vaccine immunogenicity, J Control Release. 2021 Jul. 10; 335:237-246, Mauger D M et al., mRNA structure regulates protein expression through changes in functional half-life, Proc Natl Acad Sci USA. 2019 Nov. 26; 116(48):24075-24083, Hassett K J et al., Optimization of Lipid Nanoparticles for Intramuscular Administration of mRNA Vaccines., Mol Ther Nucleic Acids. 2019 Apr. 15; 15:1-11.

There is also provided herein an IdeS polypeptide wherein said polypeptide is present in a pharmaceutical composition, optionally wherein said composition further comprises a pharmaceutically acceptable excipient. Examples of pharmaceutically acceptable excipients are mentioned elsewhere herein, but such are also known in the art and apparent to the skilled person.

In a second aspect the present invention provides a method for treating or preventing a disease or disorder mediated by pathogenic polymeric IgA complexes, wherein said disease or disorder is progressive IgA nephropathy (IgAN) or recurrent IgAN following renal transplant, wherein said method comprises the administration of an effective amount of an IdeS polypeptide to a subject.

There is also provided a method wherein said method comprises using IdeS as an induction treatment of said IgAN disease or disorder, wherein said induction treatment is followed by a subsequent one or more additional treatment(s) comprising an IdeS polypeptide and/or other IgAN specific treatments. Said additional treatments are described elsewhere herein. There is also provided a method wherein said other IgAN specific treatment comprises using an IdeS polypeptide in the form of an mRNA-based therapeutic agent encoding said IdeS polypeptide. There is also provided a method wherein said subject is an acute incident subject. Such a subject is described elsewhere herein.

It is shown herein that an IdeS polypeptide is capable of cleaving polymeric IgA complexes.

The treatment or preventing of a disease or disorder mediated by pathogenic polymeric IgA complexes as disclosed herein involves inhibition of complement activation.

The polymeric IgA complexes may be anti-glycan (IgA1) IgG, IgG-GdIgA1 immune complexes. The structure of such anti-glycan (IgA1) IgG, IgG-GdIgA1 immune complexes have been extensively studied in the art.

Herein, the treatment or prevention of IgAN as defined herein includes disintegration of the IgA complexes. As mentioned herein, this disintegration surprisingly renders the IgA complexes non-pathogenic.

The treatment of prevention of a disease mediated by polymeric IgA complexes involves administration of one or more IdeS polypeptides. Such administration may be a single administration of IdeS in an effective amount or it may be a regime of repeated administration of IdeS over a period of time. In a treatment regime of repeated administration of IdeS, the amount of IdeS administered may be the same or it may change from one administration to the next. E.g. the amount of IdeS administered to a patient-may decrease or increase over time.

Pharmaceutical composition comprising IdeS and various carriers, diluents, preservatives etc are known in the art, see eg. EP1901773B1.

There is also provided an IdeS polypeptide as disclosed herein for use in a method for the treatment or prevention of Focal Segmental GlomeruloSclerosis (FSGS). This is a condition very often associated with progressive disease or recurrent disease in renal transplants. Such a disease condition has a similar behavior to IgAN disease, wherein said disease arises due to IgG complex formation.

There is also provided herein a method for treating or preventing Focal Segmental GlomeruloSclerosis (FSGS) in a subject, said method comprising the administration of an effective amount of an IdeS polypeptide as disclosed herein to a subject in need thereof.

### Itemized Various Embodiments Within the Present Disclosure

Embodiment 1: An IdeS polypeptide for use in a method of treating or preventing a disease mediated by polymeric IgA complexes.

Embodiment 2: The IdeS polypeptide according to embodiment 1, wherein said disease is IgA nephropathy (IgAN) or recurrence of IgA nephropathy.

Embodiment 3: The IdeS polypeptide according to any of the preceding embodiment, wherein said treating or preventing a disease mediated by polymeric IgA complexes involves inhibition of complement activation.

Embodiment 4: The IdeS polypeptide according to any of the preceding embodiment, wherein said disease is progressive IgAN or recurrent IgAN following renal transplantation.

Embodiment 5: The IdeS polypeptide according to any of the preceding embodiments, wherein said polymeric IgA complexes are anti-glycan (IgA1) IgG, IgG-GdIgA1 immune complexes.

Embodiment 6: The IdeS polypeptide according to any of the preceding embodiments wherein said treatment or prevention includes disintegration of the IgA complexes.

Embodiment 7: A method for treating or preventing a disease mediated by polymeric IgA complexes which comprises the administration of an effective amount of an IdeS polypeptide to a subject.

Embodiment 8: The method according to embodiment 7, wherein said administration of IdeS causes inhibition of complement activation.

Embodiment 9: The method according to any of embodiments 7-8, wherein said disease is IgAN or recurrence of IgAN.

Embodiment 10: Use of an IdeS polypeptide for the cleavage of polymeric IgA complexes.

Embodiment 11: The use according to embodiment 10, wherein said cleavage renders said polymeric IgA complexes non-pathogenic.

Embodiment 12: The use according to any of embodiments 10-11, wherein said IgA complexes are anti-glycan (IgA1) IgG, IgG-GdIgA1 immune complexes.

The present invention is now illustrated by the following experimental section but is not intended to be limited thereto.

### EXAMPLES

### Example 1—IdeS Digestion of IgA/IgG Immune Complexes in Human Serum

IgA 1 Purification from Blood

20-40 mls of blood was collected following written consent from acute incident IgAN patients referred to the clinic. The blood was allowed to clot on the bench for 30 minutes before centrifugation at 2000×g, for 10 minutes, at room temperature. IgA1 was isolated from the serum by jacalin agarose affinity chromatography. After isolation, the IgA was concentrated to 1 mg/ml and stored at −20° C.

### IdeS Digestion

IdeS digestion of purified IgA was performed as instructed.

A time course experiment was performed in which 5 μg of the IdeS solution was added to 1 mg of IgA and aliquots of the digest reaction were collected at 5, 15, 30, 60 and 80 minutes. 1 μl of 0.1 mM PMSF inhibitor was added on collection of the aliquot all samples were stored at −20° C. until needed.

In parallel, a concentration gradient experiment was conducted in which 1 mg of IgA was treated with 25 μg, 55 μg and 60 μg of IdeS for 80 minutes at room temperature. 1 μl of 0.1 mM PMSF inhibitor was added on collection of the aliquot all samples were stored at −20° C. until needed.

For comparison an undigested 1 mg IgA sample was separated and stored alongside the IdeS digests.

5 μl aliquots of the digestion mixes were mixed with either 40 μl of western blot reducing buffer, containing 10% 2-β mercaptoethanol, or non-reducing sample buffer. The aliquots in reducing sample buffer were boiled at 98° C. for 20 minutes. 10 μl of each sample/loading buffer mix were loaded onto an SDS PAGE alongside a molecular weight marker and western transferred and immunoblotted using 2.5% BSA blocking solution and a HRP conjugated polyclonal rabbit anti human IgG heavy chain antibody (1:2000) (Dako) or a HRP conjugated polyclonal rabbit anti human IgA heavy chain antibody (1:2000) (Dako).

### IgA Size Fractionation by Gel Filtration/FPLC

0.5 ml of IgA (1 mg/ml) was incubated with 60 μg of IdeS for 2 hours at room temperature. 1 μl of 0.1 mM PMSF inhibitor was added at the end of the incubation. The entire reaction mixture was loaded onto a HiLoad 16/600 Superdex column 200 pg (GE) and run with phosphate buffered saline (PBS). For comparison 0.5 ml of IgA (1 mg/ml) was also fractionated in the same manner.

Non-Reduced SDS PAGE with IgG Immunoblotting (FIG. 3)

The undigested IgA sample contained IgG, mainly in the form of high and very high molecular weight complexes, presumably complexed with IgA. Digestion with IdeS resulted in a decrease in the very high and high molecular weight IgG complexes both in a time and concentration dependent manner. Following IdeS treatment three major new protein bands appeared which likely represent the F(ab′)2 IgG fragment (˜105 kDa) and a gamma heavy chain fragment (˜30 kDa). An additional high intensity band at ˜50 kDa was also noted which likely represents an IgG containing complex.

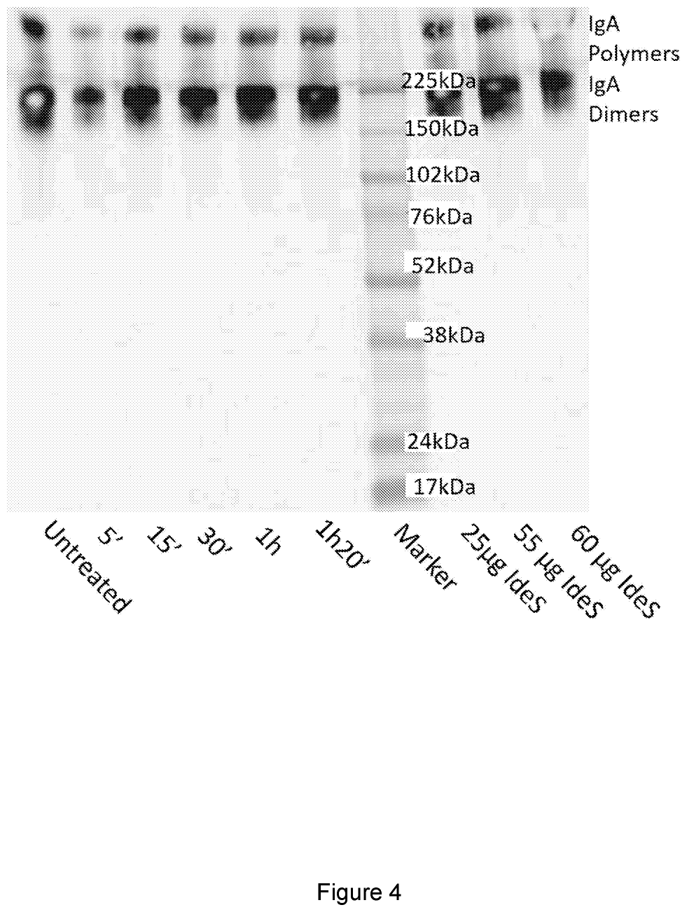

Non-Reduced SDS PAGE with IgA Immunoblotting (FIG. 4)

Digestion of the IgA sample with IdeS did not appreciably change the pattern of IgA polymers in the IgA sample. No low molecular weight IgA-IgG containing fragments were present in any of the digests implying that the high intensity band at ˜50 kDa noted on the IgG immunoblot is does not contain a detectable IgA fragment.

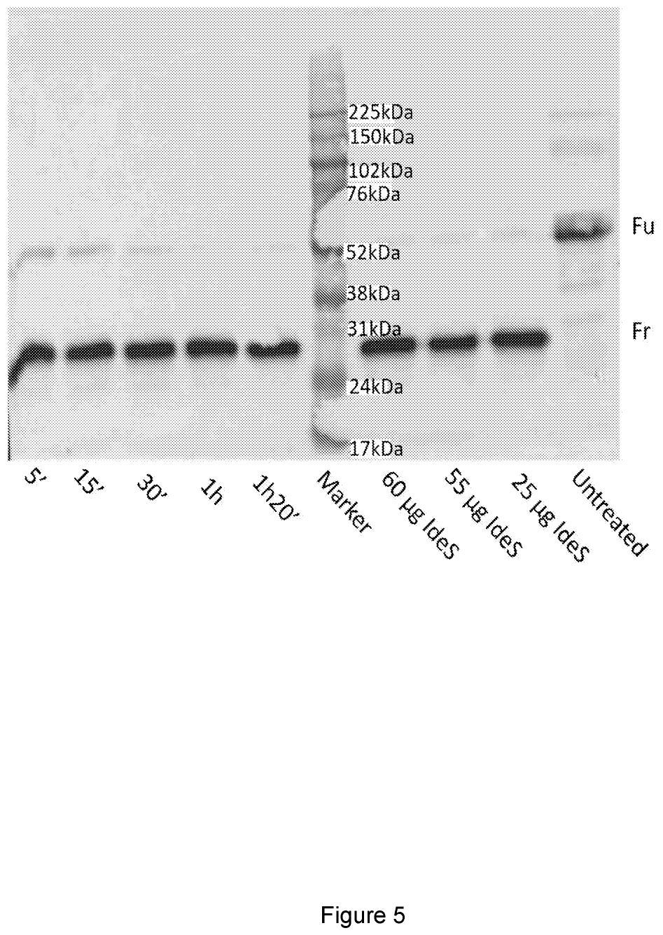

Reduced SDS PAGE with IgG Immunoblotting (FIG. 5)

On reduction the IgG within the undigested IgA sample resolved into a single band consistent with the intact gamma heavy chain. The intact gamma heavy chain was present in all IdeS digests suggesting that not all IgG was digested within the IgA/IgG complexes after IdeS treatment, although digestion with IdeS resulted in a decrease in the amount of intact gamma heavy chain both in a time and concentration dependant manner. In parallel with the non-reduced gel there was a high intensity gamma heavy chain fragment (˜30 kDa). The high intensity band at ˜50 kDa noted on the non-reduced gel was not seen following reduction suggesting that this band is an IgG fragment-containing protein complex.

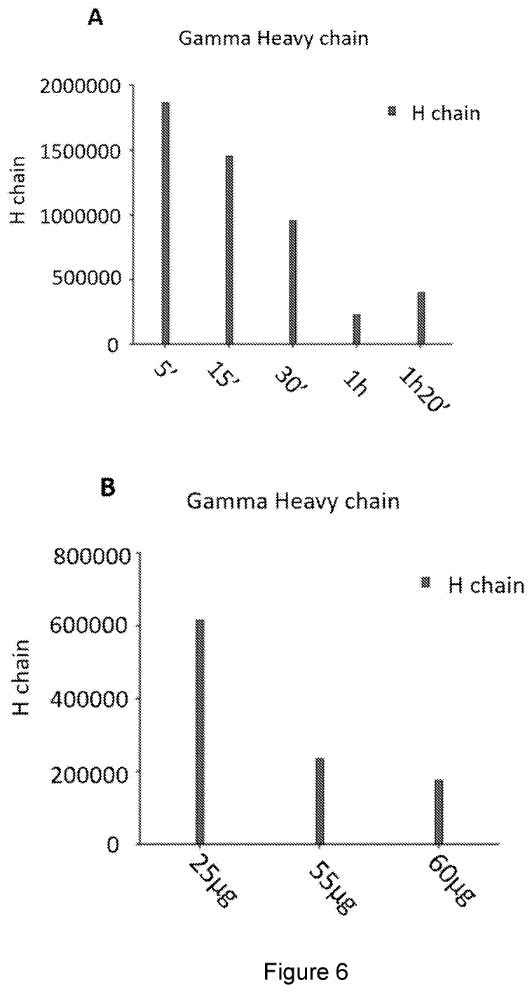

Densitometric analysis of the intact gamma heavy chain band confirmed a time and concentration dependant increase in IdeS digestion of IgG within the IgA sample (FIG. 6).

### IgA Size Fractionation of Intact and IdeS Digested IgA by Gel Filtration/FPLC.

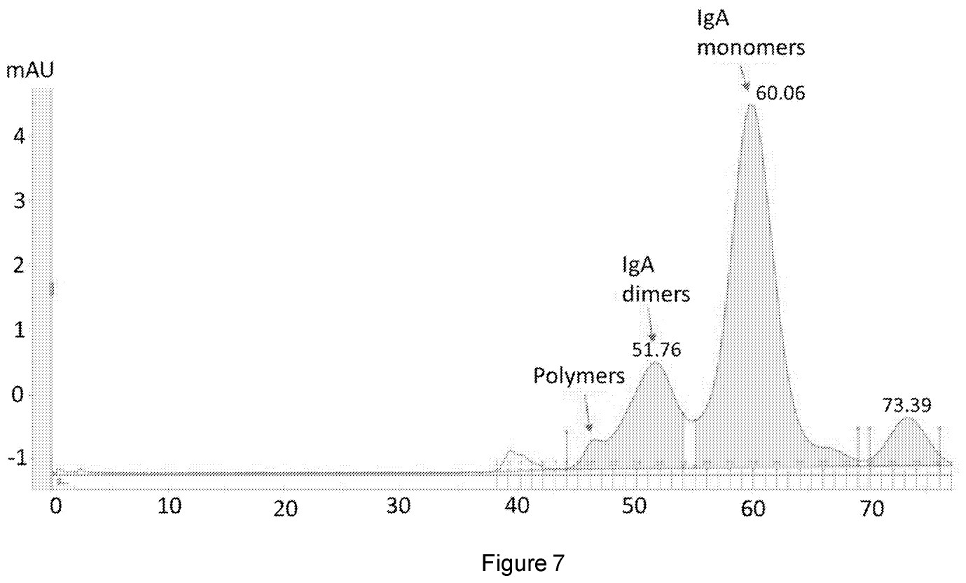

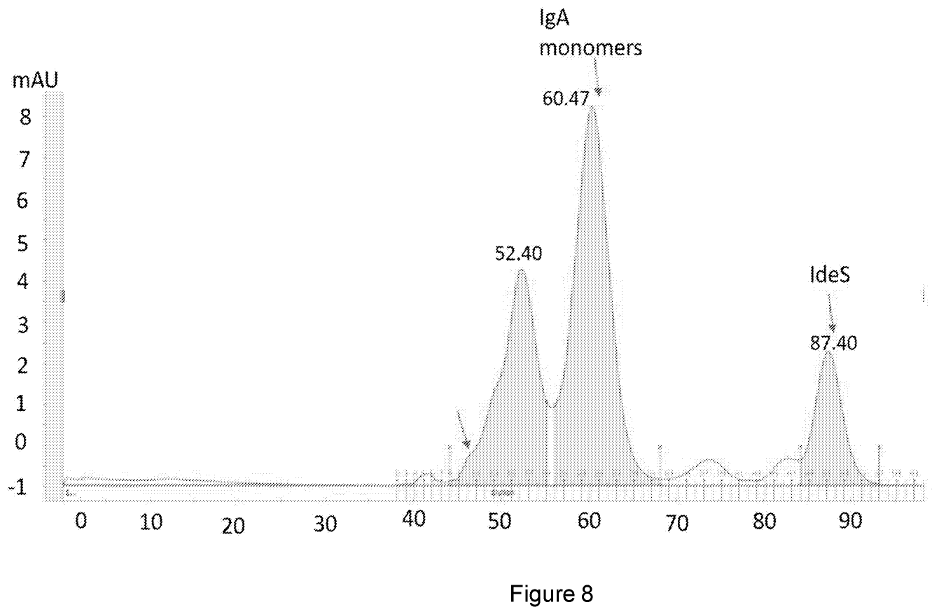

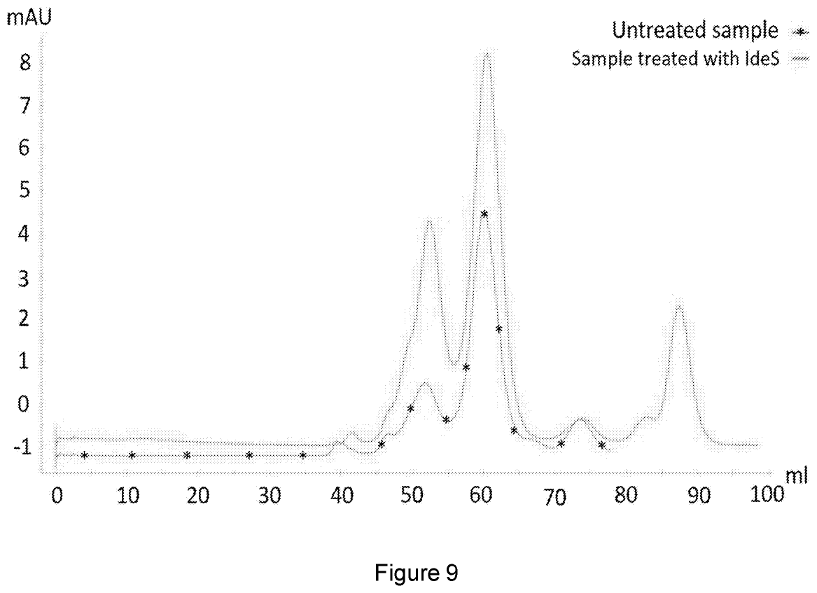

To determine the impact of IdeS digestion on the high molecular weight IgA immune complex composition of human serum we performed gel filtration by FPLC on a Superdex column (FIGS. 7, 8 and 9).

### Conclusion

Treatment of purified serum IgA with IdeS resulted in digestion of IgG, predominantly in high molecular weight complexes, which increased with incubation time and enzyme concentration.

### Example 2—IL6 Effects

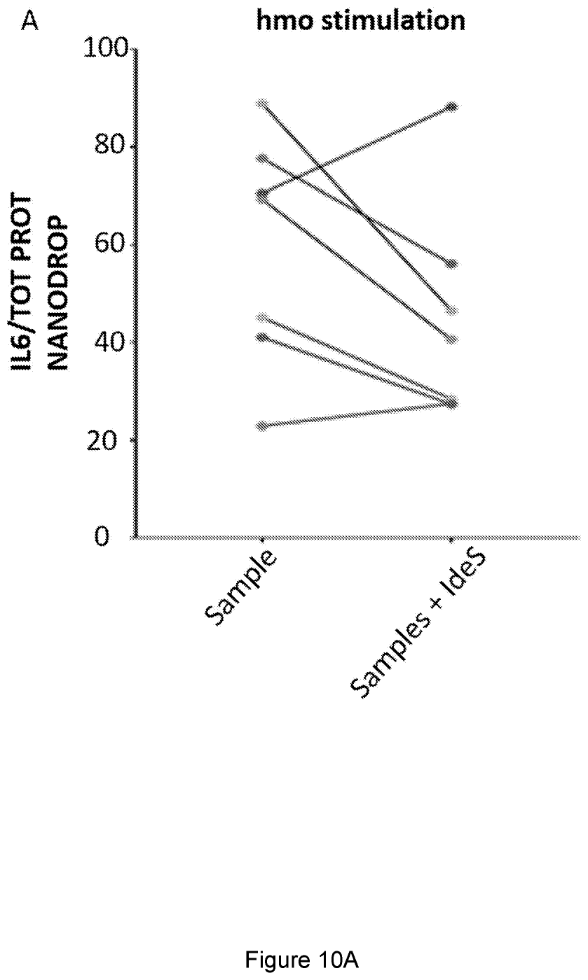

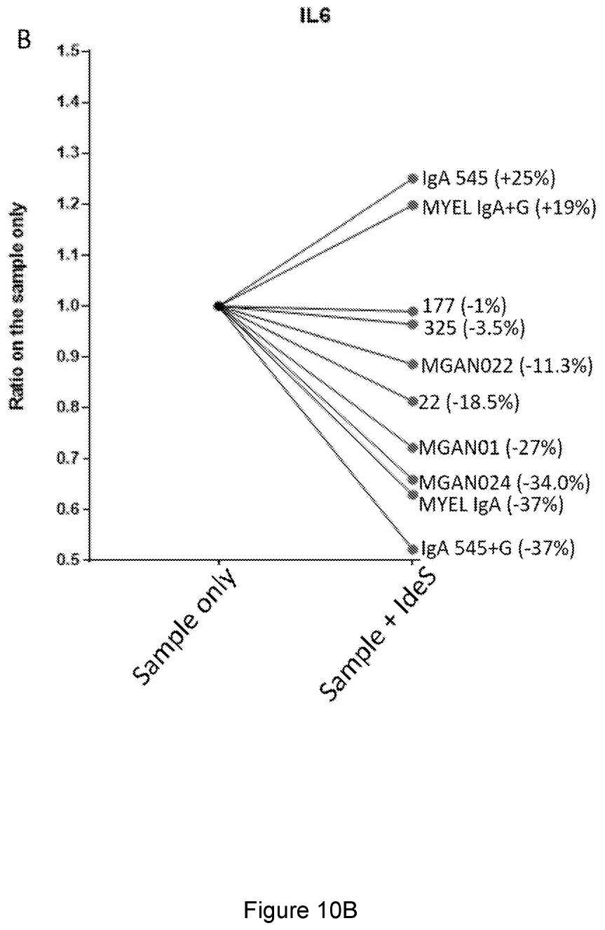

Two different human mesangial cell lines were exposed to IgA samples in a concentration of 100 ug/ml where the IgA samples were either undigested or digested with 60 ug of IdeS (imlifidase). The results are shown in FIG. 10.

The treatment with IdeS resulted in a clear reduction in the ability of IgA immunecomplexes to activate human mesangial cells in vitro as assessed by the generation of IL6 (FIG. 10).

1. An IdeS polypeptide for use in a method of treating or preventing a disease mediated by pathogenic polymeric IgA complexes in a subject, wherein said disease or disorder is progressive IgA nephropathy (IgAN) or recurrent IgAN following renal transplantation.

2. The IdeS polypeptide for use according to claim 1, wherein said method comprises using said IdeS polypeptide as an induction therapy of said IgAN disease or disorder, wherein said induction therapy is followed by a subsequent one or more additional treatment(s) comprising an IdeS polypeptide and/or other IgAN specific treatments.

3. The IdeS polypeptide for use according to claim 2, wherein said IgAN specific treatment is selected from the group consisting of Renin-angiotensin system (RAS) blockage, corticosteroids such as budesonide, pulse therapy comprising cytostatic compounds, immunosuppressive treatments, such as immunosuppressive treatments comprising mycophenolate mofetil (MMF), calcineurine inhibitors, anti-CD20 antibodies and other treatments targeting IgG immune complex formation, such as treatments targeting FcRN interactions to prevent the formation of IgG immune complexes.

4. The IdeS popypeptide for use according to claim 2 or 3, wherein said additional IgAN treatment comprises using an IdeS polypeptide in the form of an mRNA-based therapeutic agent.

5. The Ides polypeptide for use according to any one of the preceding claims, wherein said subject is an acute incident subject.

6. The IdeS polypeptide for use according to any one of claims 1 to 5, wherein said subject is at risk of developing Chronic Kidney Disease (CKD) or End Stage Renal Failure (ESRD).

7. The IdeS polypeptide for use according to any one of claims 1 to 6, wherein said IdeS polypeptide comprises an amino acid sequence according to SEQ ID NO: 1, or a functional fragment or variant thereof.

8. The IdeS polypeptide for use according to any one of claims 1 to 7, wherein said IdeS polypeptide is provided to said subject in the form of an mRNA-based therapeutic agent.

9. The IdeS for use according to any one of claims 1 to 8, wherein said IdeS polypeptide is present in a pharmaceutical composition, optionally wherein said composition further comprises a pharmaceutically acceptable excipient.

10. A method for treating or preventing a disease or disorder mediated by pathogenic polymeric IgA complexes in a subject, wherein said disease or disorder is progressive IgA nephropathy (IgAN) or recurrent IgAN following renal transplantation, said method comprising the administration of an effective amount of an IdeS polypeptide to a subject in need thereof.

11. The method according to claim 10, wherein said method comprises using IdeS as an induction therapy of said IgAN disease or disorder, wherein said induction treatment is followed by a subsequent one or more additional treatment(s) comprising an IdeS polypeptide and/or other IgAN specific treatments.

12. The method according to claim 11, wherein said IgAN specific treatment is selected from the group consisting of Renin-angiotensin system (RAS) blockage, corticosteroids such as budesonide, pulse therapy comprising cytostatic compounds, immunosuppressive treatments, such as immunosuppressive treatments comprising mycophenolate mofetil (MMF), calcineurine inhibitors, antiCD20 antibodies and other treatments targeting IgG immune complex formation, such as treatments comprising FcRN interactions to target the formation of IgG immune complexes.

13. The method according to claim 11 or 12, wherein said other IgAN specific treatment comprises using an IdeS polypeptide in the form of an mRNA-based therapeutic agent, said mRNA-based therapeutic agent encoding said IdeS polypeptide.

14. The method according to any one of claims 10 to 13, wherein said subject is an acute incident subject.

15. The method according to any one of claims 10 to 14, wherein said subject is at risk of developing Chronic Kidney Disease (CKD) or End Stage Renal Failure (ESRD).

16. The method according to any one of claims 10 to 15, wherein said IdeS polypeptide comprises an amino acid sequence according to SEQ ID NO:1, or a functional fragment or variant thereof.

17. The method according to any one of claims 10 to 16, wherein said IdeS polypeptide is administered to said subject in the form of an mRNA-based therapeutic agent encoding said IdeS polypeptide.

18. The method according to any one of claims 10 to 17, wherein said IdeS polypeptide is present in a pharmaceutical composition, optionally wherein said composition further comprises a pharmaceutically acceptable excipient.
